SEQUENCE LISTING (37 sequences)

SEQ ID NO: 1 - DNA, 3720 bp, Mus musculus
  gene (1)...(3720)
actagtcttcaagattactcaagactcaatactcaaaaagtcatgtttaatagggaatttaactttttcctttgtgggtaattatttcatttaaattacagtagtttttcttttctaatttttatgaaagaactacaaagtaagctagaagaaataagaatatttttgttattgatctaatctgccaatacagatcattaaatcaaagaaaatgaagataacaatcaaactgaggaagaaaaggagatagttatggaatttgacaggcaactggaaacatgcttgctaaaactgtctcttgcattttgttcctctaacctaaataacagaaggagcaaaaggaatctcttagtctctctatgtcacaatggtgcctttaggcttcattgtatttctctggttgtatttgattttcaaaaatgggaccaacagttgtacacatatagcatatatgtcagttgtgatggacacacatacagcatatatgtcagttgtgatggacacacatacagcatatatgtcagttgtgatgggcacacatacagcatatatgtcagttgtgatggacccacatacagcatatatgtcagttgtgatggacacacatacagcatatatgtcagttgtgatgggcagtcctttctagactctccggggccctacagacagtgactgcctgtaactacgacatggttttagctgtgtaaatggcactgctattgcctttctccatttatgtcagtgttctagctgaggcttccagagaacattagacattattcaacctaatgatactttcagttatcgaaaatgattttgttatcctggactcacattcattctaatgctaaaactatgttttaaaagacttattaaaataaaatacaatgtgttaagtaagattaaataagattaatcaggaaataaaatattttacattagcagcaatcacaatgtagtgtttaagagatcagtctcttaaagtctctatggtgagtttctatgtagctaataacctcacatcttcaatggacacaattcctctatcaaaaagaatcgtaacgggcagtggtggcacacacctttaatcccagcacttgggaggcagaggcaggtggatttctgagttcgaggccagcttggtctacagagtgagttccaggtcagccagggctacacagagaaaccctgtctcgaaaaaccaaaggaaaaaaaaaataaaaagaatcgtgtgtgtatcaatacaaaccttttcagtatattatggaaatagctgtgattaaagaaatgcaaaagaacaccggtttcattatcaaggcaaatcaggaggctcctaatttcaaagttgataggctttcctttcctagagagaaccttgtgcccactaatttaaatatcctaaggtctctgctaactcttagctgaattcagggctgttttatatttactagaatttactcttgaacgagttacatcctaacagaaaggacaaagttcaagagaaggcaaatgagtttttcaaagtaacaaaagataactttatacactatttcagaaggtctcactttagatactttccataacaaaactgtgactacatacttattctcctagtctttcttttcaagtaaaaccaatttgcaaaaaggtcttttttttttacaccaatctcactcacaggaactaaaagttcatgaatctcttcctctcaagaagtaactgatgcaaagattatgctagaaacaaagacaaaactgagcatgaaggatttaattccactataaaacgaatcttgctctctccacaaagaaacacaagtgtatgcaatctaacatactaaaccaatattctaatgtttttgatgaaggtggcctgcttttttctcaactctataaatcttcatttttactggactagaaatgcatccccctaccctctcccctgttgcacagtttgcacacctgaattttgcatggtcattctaatctaatcgcccccgacctcttcagaggctaaattagcatagtttctaggcagagagattctaggacaggctgtggattgagtcagaggcacctaaacagctttcagggtggtctctccttgcaagcggagcctccctttttccctttggtatttgggagactcatcaaggatccaagaggatgcacgcgggaaccactgctggctcctgccgccttaaaaataaaccatcgctcctcttggagctgcgctttagctcgaaaaaggcttcccctcgcacgctgatttgatcttcagtgccagcccatgaggtgtgccgcgtcctcgagcctagtttacaagtgaggaaaggatcgcgtccacagacccagtcctaagggccgcccactgccaggtggaggaaggcgaatggcaggagtgccacccgcagcctctcacctggcgggccatcgagaccccggagatgggcctggcggcggcggccactcaccatctggaactccccccaagtctatgtccgccagggactgggccgggaccccagaccctgacgcacctcacgcgtccccgcccccctcacgctcaccgtcggacctcgccagacagcgtctggaagggactcagaagcttcgcgtccgccgcagcctgccggggtcacggatccttcagtcccgggcgaccgtggcggtgatcacccgaaggcccgtgagctccgggaaaaggaaaacctgctcccagtcaatcggctgacatttcccagccagccggagctccacccacttccgcctgcctgtcctcctcctcctcctcctcctccttcaatcccgcccggcgccgcagggagttgtgggtatctggagggttggtccgaaatcagagtggaagaacccaggcaggactacgagctaggctcggaactacaattcccagcaggcaacggggcgtcagaaggcggctgctggagagcgggatgcagaactccgcgcgcgcggtggtgatggaacgttgctagttcgcgggtccaactgctggggccccggggatcactctaagccgagcgctaaaacaggagtctggaaggcaggcagcgcttcaattaattcaagcattcaaaactcctgaatcagtggacaccatgcataggtaaaccgattgcaaaaaaaccggacaaaacatgaatgaaccgtttgaattcttttaggccgcgaatgttgcttcttgttcaaataatctggttatgtgaatgtttgcttagagaaaaaggatggatgaatgctttagtgcgaatatcaatgtggagtttaatttggcttcctaattaagttttccttcctctactttgagtcatggtatcagtgccgtccctctttttccctccacgggaagaaattattgaggccttctgcgtgaccataaatcccaaagcctttcaaagtatatatgttcccgatttttctctttcattcttgtatttctctcgcttgtaggcacgtttaaaaaatgcagtactgtcacttgctgggacttttctccactccattctttactcttctttcatgcctaccttggaattatttaggggtgtattttcaaaaggaatagagaatgtctgcccttctgcaggaagcaccttcaggagattccggaccagagtggcaacgtcaccaccagcttcacgtggggatgggattccagcaagacttctgaactgctatcaggcatgggtgtctcggcctt

SEQ ID NO: 2 - DNA, 372 bp, Artificial Sequence
  Description of Artificial Sequence oligonucleotide
tcccgggcgaccgtggcggtgatcacccgaaggcccgtgagctccgggaaaaggaaaacctgctcccagtcaatcggctgacatttcccagccagccggagctccacccacttccgcctgcctgtcctcctcctcctcctcctcctccttcaatcccgcccggcgccgcagggagttgtgggtatctggagggttggtccgaaatcagagtggaagaacccaggcaggactacgagctaggctcggaactacaattcccagcaggcaacggggcgtcagaaggcggctgctggagagcgggatgcagaactccgcgcgcgcggtggtgatggaacgttgctagttcgcgggtccaactgctggggccccggg

SEQ ID NO: 3 - DNA, 24 bp, Artificial Sequence
  Description of Artificial Sequence oligonucleotide
taggaagcaccttcaggagattcc

SEQ ID NO: 4 - DNA, 24 bp, Artificial Sequence
  Description of Artificial Sequence oligonucleotide
acctggaaagttctcccgactaag

SEQ ID NO: 5 - DNA, 24 bp, Artificial Sequence
  Description of Artificial Sequence oligonuleotide
tgcatggtgtccactgattcagga

SEQ ID NO: 6 - DNA, 24 bp, Artificial Sequence
  Description of Artificial Sequence oligonuleotide
cattcaaaactcctgaatcagtgg

SEQ ID NO: 7 - DNA, 24 bp, Artificial Sequence
  Description of Artificial Sequence oligonuleotide
gattcctgaagacttaatgactgg

SEQ ID NO: 8 - DNA, 24 bp, Artificial Sequence
  Description of Artificial Sequence oligonucleotide
cgcgtgaggtgcgtcagggtctgg

SEQ ID NO: 9 - DNA, 24 bp, Artificial Sequence
  Description of Artificial Sequence oligonucleotide
cattctttactcttctttcatgcc

SEQ ID NO: 10 - DNA, 24 bp, Artificial Sequence
  Description of Artificial Sequence oligonucleotide
tatgttagattgcatacacttgtg

SEQ ID NO: 11 - DNA, 24 bp, Artificial Sequence
  Description of Artificial Sequence oligonucleotide
tttcctagagagaaccttgtgccc

SEQ ID NO: 12 - DNA, 24 bp, Artificial Sequence
  Description of Artificial Sequence oligonucleotide
tgtgtgccaccactgcccgttacg

SEQ ID NO: 13 - DNA, 24 bp, Artificial Sequence
  Description of Artificial Sequence oligonucleotide
agttgtgatggacccacatacagc

SEQ ID NO: 14 - DNA, 22 bp, Artificial Sequence
  Description of Artificial Sequence oligonucleotide
ctcccgactaagcttcggccga

SEQ ID NO: 15 - DNA, 25 bp, Artificial Sequence
  Description of Artificial Sequence oligonucleotide
gagcagtccatgtgggatgttctca

SEQ ID NO: 16 - DNA, 23 bp, Artificial Sequence
  Description of Artificial Sequence oligonucleotide
catccccacgtgaagctggtggt

SEQ ID NO: 17 - DNA, 20 bp, Artificial Sequence
  Description of Artificial Sequence oligonucleotide
ccaccagcttcacgtgggga

SEQ ID NO: 18 - DNA, 20 bp, Artificial Sequence
  Description of Artificial Sequence oligonucleotide
gcagtcattctttagcatga

SEQ ID NO: 19 - DNA, 12 bp, Artificial Sequence
  Description of Artificial Sequence oligonucleotide
gctccgggaaaa

SEQ ID NO: 20 - DNA, 21 bp, Artificial Sequence
  Description of Artificial Sequence oligonucleotide
aatcccgcccggcgccgcagg

SEQ ID NO: 21 - DNA, 21 bp, Artificial Sequence
  Description of Artificial Sequence oligonucleotide
aatcccacccggcacctcagg

SEQ ID NO: 22 - DNA, 21 bp, Artificial Sequence
  Description of Artificial Sequence oligonucleotide
aatcccgcccggcgccgcagg

SEQ ID NO: 23 - DNA, 21 bp, Artificial Sequence
  Description of Artificial Sequence oligonucleotide
aatccatcccggcacctcagg

SEQ ID NO: 24 - DNA, 21 bp, Artificial Sequence
  Description of Artificial Sequence oligonucleotide
aatcccgcccggcgccgcagg

SEQ ID NO: 25 - DNA, 21 bp, Artificial Sequence
  Description of Artificial Sequence oligonucleotide
aatcccgcccggatccgcagg

SEQ ID NO: 26 - DNA, 21 bp, Artificial Sequence
  Description of Artificial Sequence oligonucleotide
aatcccgcccggcgccgcagg

SEQ ID NO: 27 - DNA, 21 bp, Artificial Sequence
  Description of Artificial Sequence oligonucleotide
aatcccgcccggcgcatcagg

SEQ ID NO: 28 - DNA, 33 bp, Artificial Sequence
  Description of Artificial Sequence oligonucleotide
agagcgggatgcagaactccgcgcgcgcggtgg

SEQ ID NO: 29 - DNA, 30 bp, Artificial Sequence
  Description of Artificial Sequence oligonucleotide
agagcatgcagaactccgcacgagcggtgg

SEQ ID NO: 30 - DNA, 33 bp, Artificial Sequence
  Description of Artificial Sequence oligonucleotide
agagatggatgcagaactccgcgcgcgcggtgg

SEQ ID NO: 31 - DNA, 33 bp, Artificial Sequence
  Description of Artificial Sequence oligonucleotide
agagcgggatgcagaactccgatcgcgcggtgg

SEQ ID NO: 32 - DNA, 33 bp, Artificial Sequence
  Description of Artificial Sequence oligonucleotide
agagcgggatgcagaactccgcgatcgcggtgg

SEQ ID NO: 33 - DNA, 33 bp, Artificial Sequence
  Description of Artificial Sequence oligonucleotide
agagcgggatgcagaactccgcgcgatcggtgg

SEQ ID NO: 34 - DNA, 33 bp, Artificial Sequence; identical to SEQ ID NO: 31
  Description of Artificial Sequence oligonucleotide

SEQ ID NO: 35 - DNA, 33 bp, Artificial Sequence; identical to SEQ ID NO: 33
  Description of Artificial Sequence oligonucleotide

SEQ ID NO: 36 - DNA, 13 bp, Artificial Sequence
  Description of Artificial Sequence oligonucleotide
agttcgcgggtcc

SEQ ID NO: 37 - DNA, 13 bp, Artificial Sequence
  Description of Artificial Sequence oligonucleotide
agttaacgggtcc

(19) United States
(12) Patent Application Publication
(10) Pub. No.: US 2006/0257367 A1
(43) Pub. Date: Nov. 16, 2006

(54) Tumour-cell specific gene expression and its use in cancer therapy

(76) Inventors: Anne-Isabelle Gallani, Strasbourg (FR); Georges Imbert, Buschwiller (FR); Wilhelm Krek, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/352,053

(22) Filed: Feb. 10, 2006

Related U.S. Application Data
(62) Division of application No. 10/311,946, filed on Dec. 19, 2002, now abandoned.

(30) Foreign Application Priority Data
Jun. 23, 2000 (GB) 0015371.8

(51) Int. Cl. A61K 48/00; C12N 15/861; C07H 21/04

(52) U.S. Cl. 424/93.2; 435/456; 435/325; 536/23.2

(57) ABSTRACT

A polynucleotide comprising an Skp 2 promoter sequence is provided. The promoter is useful in tumour and cancer therapy, in particular in therapies that are intended to selectively kill or reduce the growth, division or viability of tumour or cancer cells compared to surrounding, non-tumour or non-cancer cells. Also provided are methods for screening for inhibitors of Skp2 promoter activity.

The invention relates to the field of tumour and cancer therapy. More particularly, therapies which are intended to selectively kill or reduce the growth, division or viability of tumour or cancer cells compared to surrounding, non-tumour or non-cancer cells. One aspect of the field to which the invention relates is that of selective expression of genes in tumour or cancer cells whereby tumour or cancer cells are killed, their activities inhibited or they are rendered more sensitive to existing therapeutic agents, e.g. ionising radiation. The invention also relates to nucleic acids, nucleic acid constructs, vectors and recombinant viruses comprising a promoter sequence of a gene expressed specifically or over-expressed in tumour cells or cancer cells compared to surrounding non-tumour or non-cancer cells. Methods of cancer therapy and pharmaceutical preparations for cancer therapies are also fields to which the invention relates.

In the more affluent countries of the world cancer is the cause of death of roughly one person in five. The American Cancer Society in 1993 reported that the five most common cancers are those of the lung, stomach, breast, colon/rectum and the uterine cervix. Cancer is not fatal in every case and only about half the number of people who develop cancer die of it. The problem facing cancer patients and their physicians is that seeking to cure cancer is like trying to get rid of weeds. Although cancer cells can be removed surgically or destroyed with toxic compounds or with radiation, it is very hard to eliminate all of the cancerous cells. A general goal is to find better ways of selectively killing cancer cells whilst leaving normal cells of the body unaffected.

Over past decades the understanding of the molecular basis of cancer has grown exponentially and several lines of evidence indicate that tumour development in humans is a multistep process. These steps reflect genetic alterations that drive the progressive transformation of normal human cells into highly malignant derivatives that are characterized by increased cell proliferation, decreased cell death, genomic instability and sustained angiogenesis (Hanahan D. & Weinberg R. A. (2000) Cell 100: 57-70). These insights have also suggested new therapeutic approaches that specifically target the molecular interactions and biochemical pathways that are changed in tumour cells. The challenge is to translate the growing understanding of the molecular basis of cancer into improvements in the treatment of cancer.

Tumour cells have lost the normal control of the cell cycle and so divide out of control compared to normal cells. The sub-cellular machinery which controls the cell cycle is a complex biochemical device made up of a set of interacting proteins that induce and co-ordinate the essential processes of duplication and division of the contents of a cell. In the normal cell cycle, the control system is regulated such that it can stop at specific points in the cycle. The stopping points allow for systems of feedback control from the processes of duplication or division. They also provide points for regulation by environmental signals.

Gene expression plays an integral part in cell division and its control. Loss of control of cell division may in certain instances have its origin in an alteration in gene expression. Analysis of genetic alterations in tumour or cancer cells has revealed many genes which encode proteins involved in the control of cell division in some way.

Oncogenes are one family of such genes. Oncogenes are either expressed in cancer cells in a mutated form or they are over-expressed in normal form. The products of such oncogenes promote cell proliferation. The non-mutated or normally expressed version of an oncogene is known as a proto-oncogene and this is expressed in normal cells and encodes a constituent protein of the normal cellular machinery.

Another kind of gene product connected with cancer is that expressed by tumour-suppressor genes and the gene products serve to restrain cell proliferation. One example is the retinoblastoma gene product (pRB). Mutation of a tumour-suppressor gene or loss of function of the gene product results in a loss of the normal control of proliferation and the cell divides out of control.

The study of tumour cells and their oncogenes or tumour-suppressor genes has helped to show how growth factors regulate cell proliferation in normal cells through a complex network of intracellular signalling cascades. These cascades ultimately regulate gene transcription and the assembly and activation of the cell cycle control system. Recent advances in molecular oncology have led to the identification of proteins that are quantitatively or qualitatively altered in cancer cells. In many instances these proteins participate in the key pathways that are important in the control of cell proliferation. The identification of these molecular pathways assist in an identification of putative therapeutic targets. Moreover the targets can be shown to have a genetic basis in the sense that they are recurrently altered in tumours and play a causal role in maintenance of the malignant phenotype. Specific therapies can be devised against the putative targets to control or eliminate cancer cells while sparing nontumorigenic counterparts.

The normal cell cycle control system is based on two main families of proteins. The first is the family of cyclin-dependent protein kinases (Cdk) of which there are a number of varieties, e.g. Cdk 1 and Cdk 2. Cdk phosphorylates selected proteins at serine and threonine residues. The second sort of protein is a family of specialised activating proteins called cyclins that bind to Cdk molecules and control their ability to phosphorylate targets. Cyclins themselves undergo a cycle of synthesis and degradation within each division of the cell cycle. There are a variety of species of cyclin, e.g. cyclin A and cyclin B.

Chao Y et al (1998) Cancer Research 58: 985-990 report a correlation between over-expression of cyclin A in patients and proliferative activity of tumour cells compared to those patients expressing a normal cyclin A level. Patients over-expressing cyclin A had a shorter medium disease-free survival time than those who did not over-express. Chao et al (1998) also report that a cyclin A-interacting protein (Skp 2) did not exhibit the same correlation with tumour cell activity as cyclin A when over-expressed. Chao et al (1998) remark on how expression of Skp 2 appears to be involved in the control of cell cycle progression but caution that the actual biochemical function of Skp 2 is still not known.

In a more recent paper, Chao Y et al (1999) Cancer Letters 139: 1-6 conclude that cyclin A may provide a useful target for the exploration of new anti-hepatocelluar carcinoma (HCC) therapeutics. In particular, Chao et al (1999) showed that an over-expression of cyclin A in HCC cells could be inhibited with antisense mRNA for the cyclin A gene. Although an over-expression of Skp 2 is apparently also associated with HCC cell proliferation, Chao et al (1999) indicate that the biochemical function of Skp 2 remains unknown. For example, the results of an experiment seeking to block over-expression of Skp 2 using antisense mRNA suggests that abnormal Skp 2 expression has no direct correlation with HCC proliferation.

The activity of CDK is subject to regulation in the cell and a CDK inhibitor protein (p27) has been identified. In normal cells p27 has been shown to regulate the action of CDK's that are necessary for DNA replication. Levels of p27 are found to be high in quiescent cells and low in cells stimulated to divide. p27 appears to act as a brake on cell division by inhibiting activated CDK which itself drives cells to divide. A reduction in the level of p27 frees activated CDK from inhibition and drives cells to divide. A reduction in the level of p27 frees activated CDK from inhibition and drives cells to divide. Consistent with this activity of p27 is the way in which its destabilisation correlates generally with tumour aggressiveness and poor prognosis for cancer patients.

The cell cycle control system is a dynamic system and p27 itself does not remain at a constant level in the cell. The level is different depending on the point in the cell cycle. Lower levels of p27 arise due to breakdown via ubiquitination and subsequent proteasome-mediated degradation. A requirement for ubiquitin-mediated degradation of p27 is phosphorylation of the threonine residue 187 (T187) by activated CDK.

The ubiquitin system of intracellular protein breakdown is a mediator of normal biological function and certain abnormalities associated with tumour development (Koepp D. M. et al., (1999) Cell 97: 431-434; and Krek W. (1998) Curr. Opin. Genet. Dev. 8: 36-42). The enzymes needed for ubiquitination of phosphorylated p27 are not known, although from knowledge of ubiquitination in systems such as yeast it is expected that there may be a human ubiquitin-protein ligase (E3) specific for p27. The F-box/leucine-rich-repeat-containing oncoprotein Skp 2 appears to have a role in neoplastic signalling. Skp 2 appears to function as a substrate-specific receptor of ubiquitination machines (the SCF ubiquitin protein ligases, that target proteins for ubiquitination and degradation (Lisztwan J. et al., (1998) EMBO J. 17: 368-383). Skp 2 has been shown to play an important role in the regulation of the dynamics of cell cycle entry and exit in mammalian cells and to target p27^{KiP1}, a cyclin-dependent kinase inhibitor and multiple-tissue type tumour suppressor, for ubiquitination and degradation. In doing so, Skp 2 promotes unconstrained cell cycle progression (Sutterluty H. et al., (1999) Nature Cell Biology 1(4): 207-214).

More particularly, Sutterluty H et al (1999) Nature Cell Biology 1: 207-214 report that Skp 2 promotes the degradation of p27 in cells via the ubiquitination pathway. Skp 2 is confirmed as being a member of the F-Box-Protein (FBP) family. Skp 2 appears to be a p27 specific receptor of an Skp 1, Cull (Cdc53), F-Box Protein (SCF) complex. Such complexes are known in yeast and act as ubiquitin-protein ligases (E3) in which the FBP subunit has specificity for the substrate for ubiquitination. E3 facilitates the transfer of an activated ubiquitin molecule from a ubiquitin-conjugating enzyme (E2) to the substrate to be degraded. Similarly, in humans there are SCF complexes and Skp 2 is an FBP which has an ability to interact specifically with p27 and which appears to be essential in the ubiquitin-mediated degradation of p27. Both in vivo and in vitro, Skp 2 is found to be a rate-limiting component of the cellular machinery which ubiquitinates and degrades phosphorylated p27.

Sutterluty et al (1999) supra found that a mutant of Skp 2 which does not assemble into an SCF complex was defective in promoting the elimination of ectopically produced wild-type p27. Also, mutant Skp 2 produced an activation of cyclin-E/A associated kinases and an induction of the S phase. Skp 2 also appears to have an independent binding site for CDK and activated CDK is involved in the phosphorylation of the T187 residue of p27. Sutterluty et al (1999) supra also note how normal Skp 2 induces an accumulation of cyclin A protein, even when activation of cyclin-E/A-dependent kinases and entry into S phase are blocked by the expression of a non-degradable p27 mutant. What is concluded is that Skp 2 up-regulates cyclin A and independently of this down-regulates p27. The mechanism by which Skp 2 up-regulates cyclin A is not known. There is a suggestion that observed increased levels of Skp 2 in transformed cells might contribute to the process of tumourigenisis, at least partly, by causing an increase in the rate of degradation of the tumour suppressing agent p27. A lack of p27 expression correlates with a reduced disease-free survival of patients with colorectal and breast cancer. Also, p27 has been found to be haplo-insufficient for tumour suppression.

Carrano A. C. et al (1999) Nature Cell Biology 1: 193-199 report how Skp 2 interacts physically with phosphorylated p27 both in vitro and in vivo. Whilst every component of the ligase machinery required for p27 ubiquitination remains to be discovered, Carrano et al (1999) demonstrate that Skp 2 is a part of this machinery and provides substrate recognition and specificity for p27. Antisense oligonucleotides against Skp 2 were found to decrease Skp 2 expression in cells and thereby result in increased levels of endogenous p27. Carrano et al (1999) also confirm an additional need for cyclin E-CDK 2 or cyclin A-CDK 2 for ubiquitination of p27 to take place. p27 degradation in cells appears to be subject to dual control by accumulation of both Skp 2 and cyclins following mitogenic stimulation.

Many of the genes that are altered in human cancer define molecular pathways that are central to the control of cell cycle and gene transcription. For example, most human cancers harbour mutations that directly or indirectly inactivate the retinoblastoma gene product (pRB) resulting in deregulated transit of cells through G1 phase and entry into S phase. Examples of mutations that directly or indirectly inactivate pRB include mutations affecting genes such as p16Ink4a, Cdk4 and cyclin D1, which lead to the untimely phosphorylation and hence, functional inactivation of pRB.

pRB binds to members of the E2F cell cycle-regulatory transcription factor family and in doing so converts them from transcriptional activators into transcriptional repressors (Sherr C. J. (1996) Science 274: 1672-1677). Therefore, cells lacking functional pRB are characterized by loss of pRB-E2F transcriptional repressor complexes and an excess of free E2F capable of activating transcription. Although increased levels of E2F can induce cellular proliferation in cells, several lines of evidence suggest that an increase of free E2F correlates with a vulnerability of cancer cells to die by apoptosis via p53-dependent and p53-independent pathways (Phillips A. C. et al., Genes Dev. 11: 1853-1863; Qin X.-Q. et al., (1994) Proc. Natl. Acad. Sci. USA 91: 10918-10922; Wu X. & Levine A. J. (1994) Proc. Natl. Acad. Sci. USA 91: 3602-3606). The latter property of E2F is of interest in cancer therapy because many tumours have lost p53 function (Levine, A. (1997) Cell 88: 323-331). Thus, there may be specific differences between normal and cancer cells. The identification and exploitation of these differences may provide new opportunities for selectively targeting cancer cells while leaving their normal counterparts untouched.

High levels of E2F-1 are known to favour transit through the G1 phase and entry into S phase (Johnson, D. G., et al., (1993) Nature 365: 349-352). Within S phase, E2F-1 activity is again downregulated by cyclin A-cyclin-dependent kinase(Cdk)₂ (Dynlacht B. D. et al., (1994) Genes Dev. 8: 1772-1786; Krek W. et al., (1994) Curr. Opin. Genet. Dev. 8: 36-42). Failure of cyclin A-Cdk2 to inactivate E2F-1 in S phase leads to apoptosis (Chen Y.-N. P. et al., (1999) Proc. Natl. Acad. Sci. USA 96: 4325-4329 and Krek W. et al., (1995) Cell 83: 1149-1158). Chen et al., (1999) supra suggested that tumour cells, by virtue of high levels of E2F-1 activity due to an inactivated pRB-pathway, might be uniquely sensitive to cyclin A-Cdk2 antagonists. Indeed, cell membrane-permeable peptides that block the interaction of cyclin A-Cdk2 and E2F-1 induce apoptosis in a wide variety of tumour cells but not in non-transformed cells (Chen et al., (1999) supra. Other signal transduction pathways are known to be disrupted in many types of cancer including the ones regulated by von Hippel Lindau (VHL) (Kaelin W. G. & Maher E. R. (1998) Trends Genet. 14: 423-426).

Radiotherapy is a significant curative treatment for head and neck cancer and the ultimate outcome depends on various tumour and host factors including intrinsic radiosensitivity (Perez B. (1992) “Principles and Practice of Radiation Oncology”: Lippincott, Philadelphia). There is a need for therapies which increase the therapeutic index, i.e. the window where radiotherapy kills tumour cells and yet normal tissue damage is still repairable. A small increase in radiosensitivity of the tumour can lead to large gain in terms of tumour control. Normal tissue structure often limits the total dose of radiation which can safely be given and so manipulation of tumour radiosensitivity can lead to a significant increase in tumour control (Fisher D. E. (1994) Cell 78: 539-542).

Elevated levels of E2F-1 can enhance the cytotoxic effect of ionizing radiation in vitro and in vivo in a mouse tumour model. This is in keeping with the view that manipulating intracellular levels of E2F-1 may push tumour cells towards an apoptotic pathway and increase their radiosensitivity (Pruschy M. et al., (1999) Exp. Cell Res. 10: 141-146). Similarly, overexpression of E2F-1 in glioma has been shown to trigger apoptosis and suppresses tumour growth in vitro and in vivo (Fueyo J. et al., (1998) Nature Medicine 4: 685-690).

A possible general direction for cancer therapy are strategies that allow tumour cell-selective transgene expression (Binley K. et al., (1999) Gene Therapy 6: 1721-1727; Heise C. et al., (1997) Nature Medicine 3: 639-645; and Parr M. J. et al., (1997) Nature Medicine 3: 1145-1149). Several approaches are available to deliver transgenes into a tumour in vivo, including adenovirus-based vectors (Benihoud K. et al., (1999) Curr. Opin. Biotechnol. 10: 440-447).

An object of the present invention is to improve current treatment regimes for cancer patients.

The inventors have observed that the abundance of Skp 2 is greatly increased in a wider range of tumour cell types than was hitherto suspected. The inventors have also realised for the first time that pathways which regulate Skp 2 expression may be commonly deregulated in cancer cells.

The inventors have discovered unexpectedly that a deregulation of Skp 2 mRNA expression is the cause of a deregulation of Skp 2 protein expression in cells of many cancers.

The inventors have also identified, located and sequenced the Skp 2 promoter. Surprisingly, the inventors have found a correlation between the deregulation of Skp 2 mRNA expression and Skp 2 promoter activity in tumour cells; namely that the Skp 2 promoter is mainly active in certain tumour cells but not in their normal counterpart cells.

Overall, the inventors are unexpectedly able to employ the Skp 2 promoter in a method of cancer cell specific gene expression, e.g a recombinant viral vector, more particularly an adenoviral vector, as a way of driving in vivo tumour cell specific expression of a suitable transgene, e.g. a gene causing apoptosis.

Accordingly, in a first aspect the present invention provides a polynucleotide comprising an Skp 2 promoter sequence. A polynucleotide comprising the promoter is distinct from the promoter sequence in nature in that it is in an isolated form. Differing degrees of purity may attach to the isolated form but preferably the polynucleotide is in substantially pure form. In numerical terms this can mean about 60%, preferably 75%, more preferably 90%, even more preferably 95% or 99% pure in the sense of being free of other contaminating nucleic acids or polynucleotides in a sample. Degree of purity may be ascertained in many ways but one convenient way is to perform gel electrophoresis and stain with ethiduim bromide, for example, as will be well known to the person of average skill in the art.

Polynucleotides of the invention may be synthetic or they may be obtained by recombinant means such as cloning and all of these methods will be well known to a person of average skill in the art.

The polynucleotide may comprise more than about 15 nucleotides, optionally more than about 25, optionally more than about 50, optionally more than about 100, optionally more than about 200 or optionally more than about 500 nucleotides. A maximum number of nucleotides is determined by the stability of the polynucleotide in relation to the procedures employed in its utilisation.

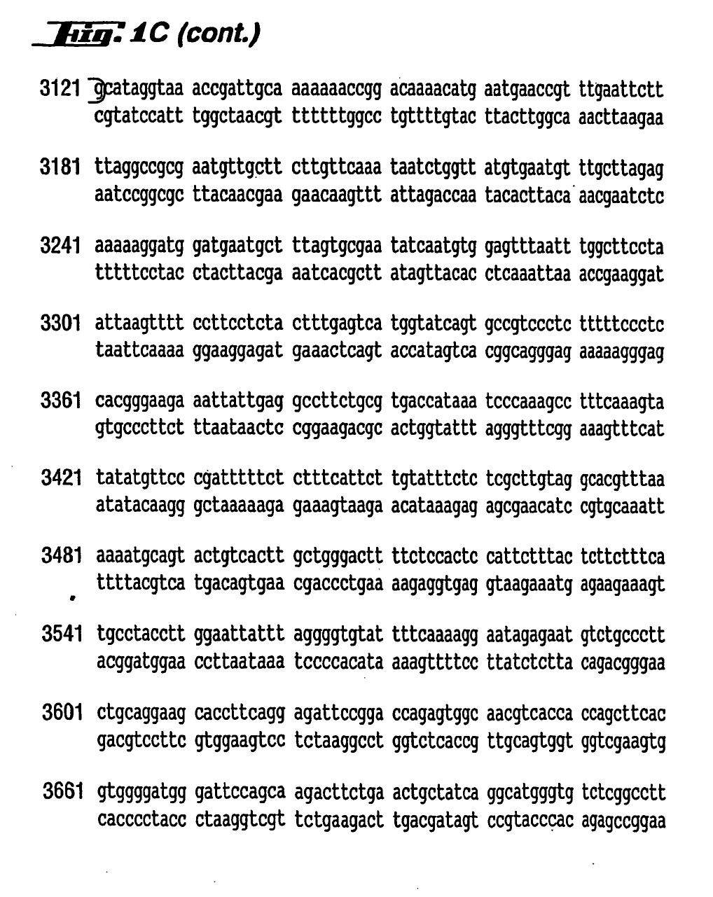

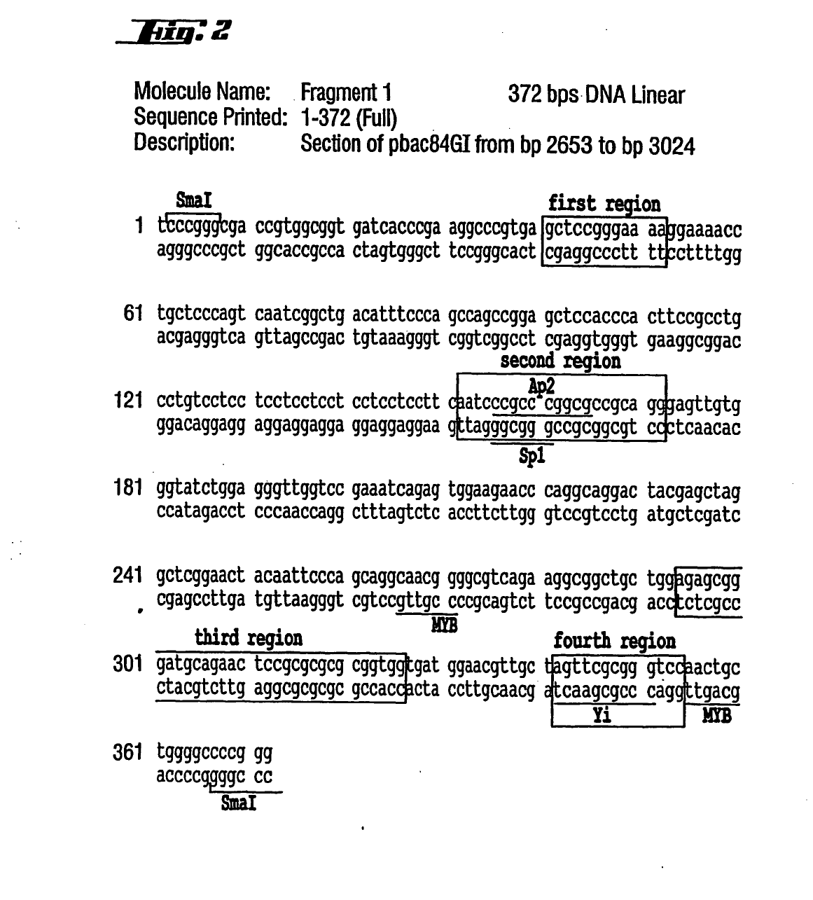

Polynucleotides of no more than 10 kb, optionally 5 kb or 1 kb are within the scope of the invention. All or part of a polynucleotide of the invention comprises the Skp 2 promoter sequence and this sequence comprises at least 15, preferably at least 25, more preferably at least 50, even more preferably at least 100, most preferably at least 350 nucleotides. In a preferred embodiment the promoter sequence comprises 360-375 nucleotides, preferably 364-370 nucleotides, most preferably 365, 366 or 367 nucleotides. A preferred promoter sequence is an Sma I fragment e.g. as shown in FIG. 3.

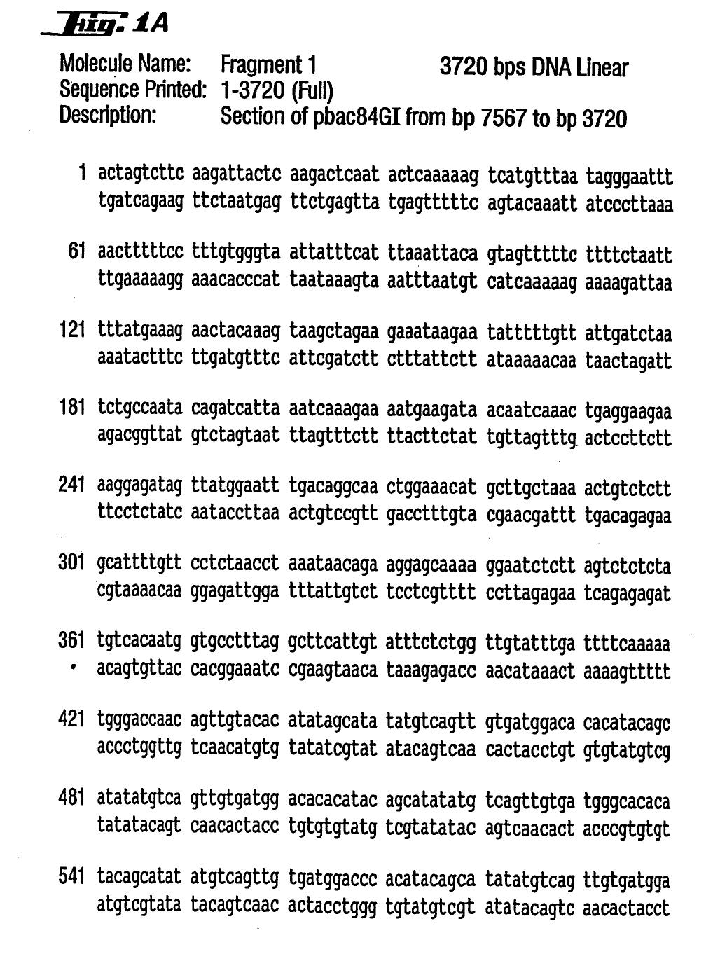

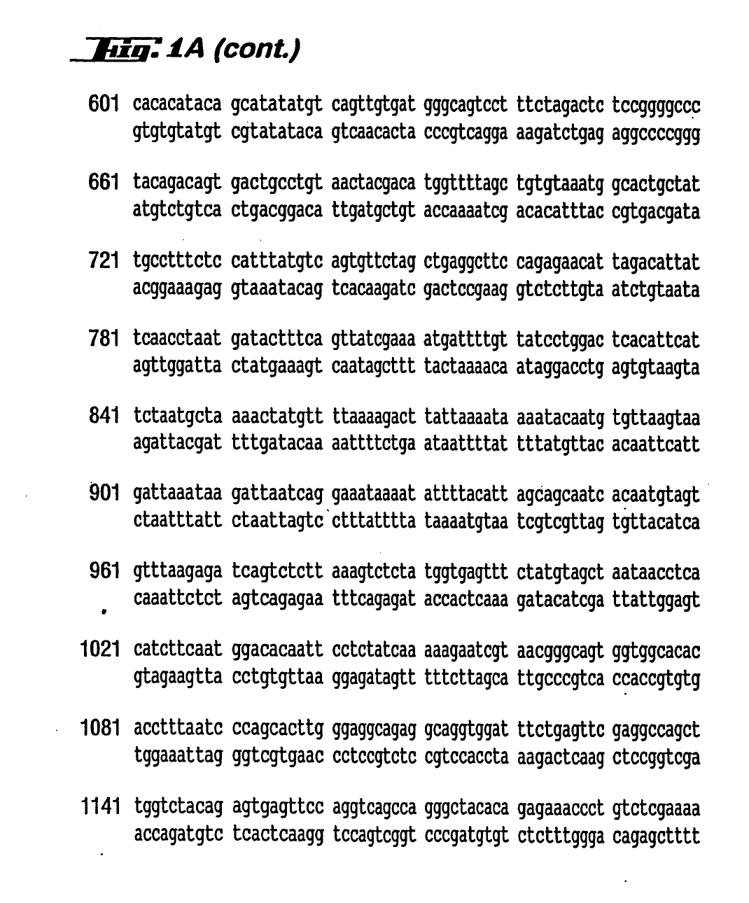

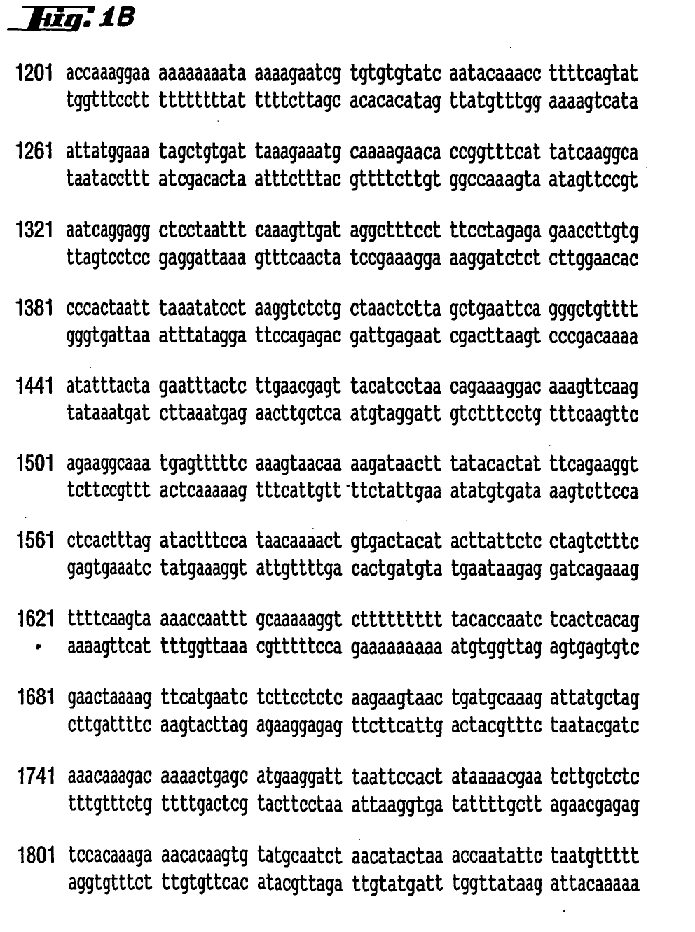

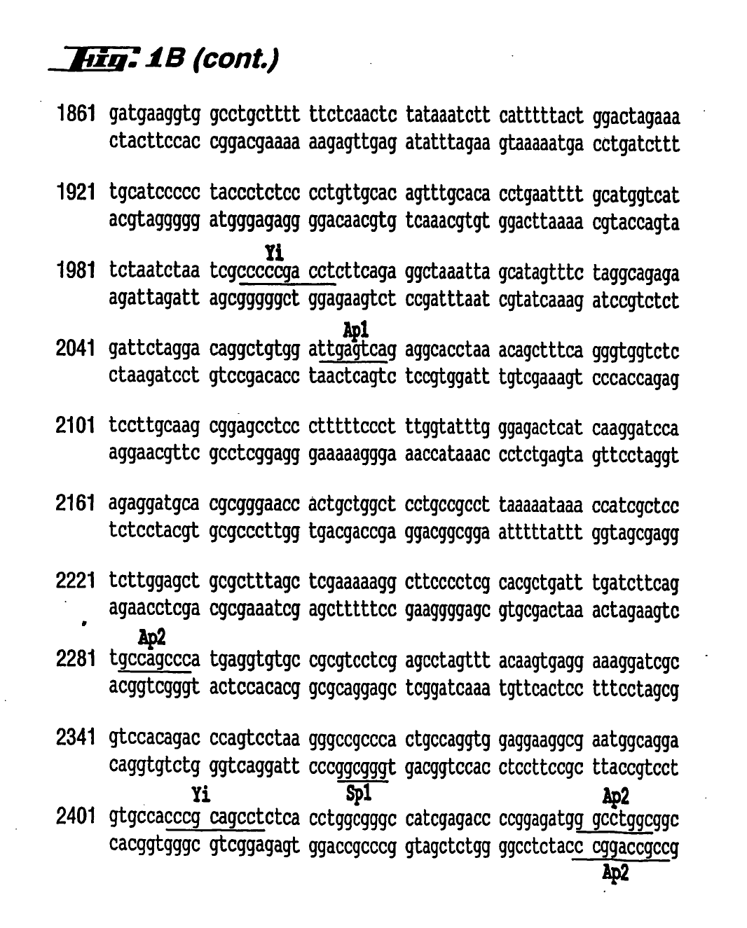

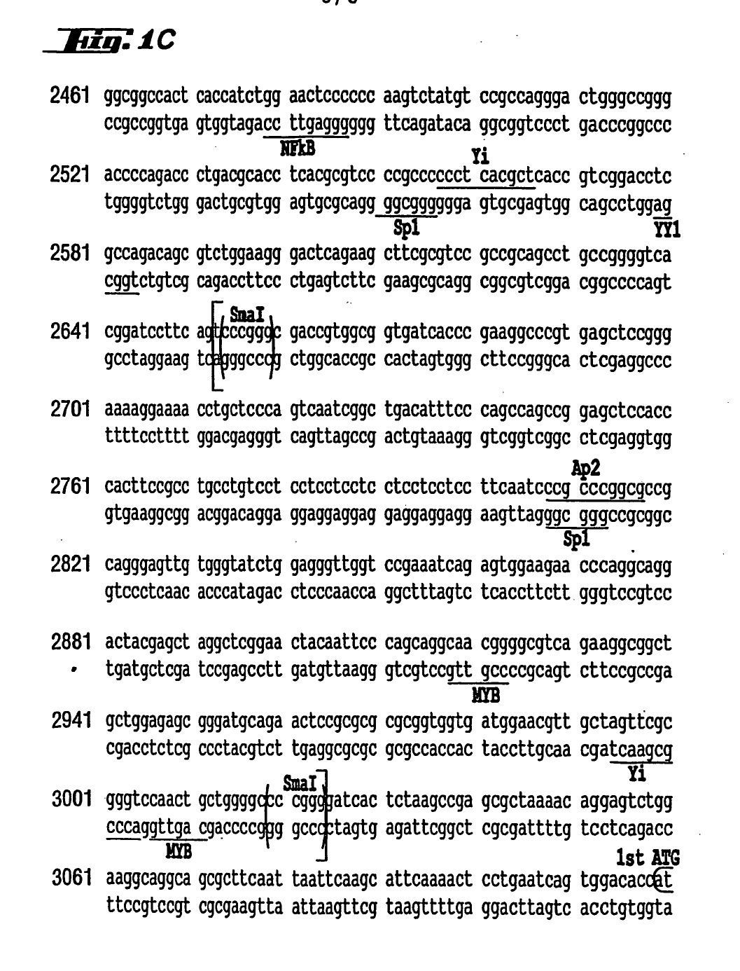

The invention therefore includes a polynucleotide as set forth in SEQ ID NO:1 (FIGS. 1A-1C) or SEQ ID NO:2 (FIG. 2) or one or more fragments thereof. Any fragments may be linked so as to be contiguous, i.e. polynucleotides of the invention including polynucleotides of SEQ ID NO:1 which have portions removed and the remaining portions are linked so as to be contiguous.

Preferred embodiments are those wherein the polynucleotide has one or more of the functional properties or activities of the Skp 2 promoter, e.g. transcription factor binding sites or regulatory factor binding sites, such as E2F1 binding sites or homologous sequences. Such binding sites can be determined empirically (by studying protein-DNA interactions), through identifying consensus sequences for binding in the promoter sequence or both. In particular, preferred polynucleotides comprise at least one of regions 1-4 (FIG. 2), preferably at least one of regions 2 and 3, or combinations thereof.

Variations in sequence compared to SEQ ID NO:1 and SEQ ID NO:2 (or portions thereof) are within the scope of the invention and the degree of variation (i.e. degree of homology) can be ascertained by performing hybridisation under stringent conditions, e.g. conditions of high salt concentration. Alternatively, the degree of homology may be ascertained by straight forward comparison of sequences and/or by known computational methods of analysis which are well known to persons of average skill in the art. The invention includes polynucleotides which may be at least 40%, preferably at least 50%, more preferably at least 60%, even more preferably at least 75%, yet more preferably at least 90% or 95%, most preferably at least 99% homologous with, or identical to all or parts of SEQ ID NO:1 or SEQ ID NO:2. As is apparent to one of ordinary skill in the art, substantial sequence variation (even up to 100%) can be tolerated in some portions of the polynucleotide sequence provided in SEQ ID NO: 1 or SEQ ID NO:2 without loss of Skp2 promoter function, whereas less variation can be tolerated in regions important for Skp2 promoter function or activity. For example, species homologues, such as human Skp2 promoter sequences, are expected to have a higher degree of homology to SEQ ID NO:1 or SEQ ID NO:2 in regions 1-4 (FIG. 2), preferably regions 2 and 3, than to sequences outside these regions.

Similarly, the artisan will recognize that the orientation of one or more regions important for Skp2 promoter function or activity to each other and transcribable sequences operably linked thereto may affect promoter activity or function. Thus, in preferred embodiments, the orientation is that found in naturally occurring sequences (e.g. SEQ ID NO:2).

The polynucleotides of the invention may be single or double stranded and may be RNA, DNA or any other form of nucleic acid, e.g. heteroduplexes. Thus, when the polynucleotides of the invention are single stranded then the invention includes the complementary strands.

Polynucleotides which are fragments of the Skp 2 promoter and which are of at least 6 to 8 nucleotides, preferably at least 10 or 12 nucleotides, but which may not have Skp 2 promoter activity, can have utility as primers and probes for use as tools in genomic analysis, diagnostic assays or simply PCR. As is apparent to one of average skill in the art, such polynucleotides (or combinations of polynucleotides) will be specific for the Skp 2 promoter sequence.

Polynucleotides of the invention also include those which are made whether wholly or partially from nucleotide analogs, e.g. non-natural synthetic nucleotide analogs, quieosine or wybutosine and acetyl-, methyl-, thio- or other chemically modified forms of the standard nucleotides which are not recognised by endonucleases. Also included are polynucleotides which comprise modified backbone analogs such as phosphorothioates and methyl phosphonates for example.

Polynucleotides of the invention may comprise or further comprise a transcribable sequence operably linked to the promoter sequence. In preferred embodiments an expressed product of the transcribable sequence causes cell death or loss of viability or inhibits cell division. The transcribable sequence may exert its effects directly or indirectly. The transcribable sequence may therefore be associated with apoptosis. The transcribable sequence may encode products which are latent but which can be activated in some way, or encode products which act on a substrate, e.g. pro-drugs which are converted to an active pharmaceutical agent. In another example, the transcribable sequence may encode thymidine kinase (TK) and upon administration of gancyclovir the TK expression cells are selectively destroyed. In preferred embodiments the transcribable sequence may be a pro-apoptotic gene. In other preferred embodiments there is an advantage in the synergistic relationship between irradiation of cells and the expression product of the expressed sequence, particularly the product of the pro-apoptotic gene in suitably transformed cells.

Other preferred expressed products of the transcribable sequence are toxins. Yet further preferred expressed products of the transcribable sequence are those which increase the sensitivity of tumour cells to radiotherapy or chemotherapy.

Other useful embodiments may have a transcribable sequence which encodes a reporter protein or peptide. The reporter may be selected, for example, from one or more of an enzyme, a ligand binding protein, a fluorescent protein or a phosphorescent protein. The reporter protein may be selected from firefly luciferase, beta-glucuronidase, chloramphenicol acetyl transferase, green fluorescent protein, enhanced green fluorescent protein or other fluorescent proteins or human secreted alkaline phosphatase. Advantageously, the presence of a visible marker in tumour cells can guide a surgeon seeking to excise those cells from a patient, e.g. by ablation of tumour cells with a laser. The marker protein can be selected so as to be tuned to the frequency of the laser light (or vice versa) so that the tumour cells light up and can be readily identified.

In further embodiments, the transcribable sequence can be an antibody fragment or marker protein, preferably a cell surface marker. Such markers can serve as targets for antibodies specific to the marker which are coupled to cell killing agents or physical markers, e.g. fluorescent proteins to help guide a surgeon seeking to excise tumour cells from a patient.

Another preferred embodiment comprises a pro-apoptotic gene as the transcribable sequence.

In particular embodiments the transcribable sequence may be an antisense sequence.

Another aspect of the invention is a polynucleotide antisense to any of the polynucleotides as hereinbefore described.

In a further aspect of the invention there is provided a genetic construct comprising a polynucleotide as described herein. Genetic constructs usually provide intermediates in the construction of expression plasmids or minigenes, for example. Suitable constructs may include linkers, restriction sites or selection markers.

In another aspect the invention provides a plasmid comprising a polynucleotide as described herein. Plasmids can provide suitable means of cloning polynucleotides of the invention or may provide convenient vehicles for achieving expression in cells or cell free systems.

Particularly useful are vectors comprising polynucleotides as described herein. The vectors permit transfection and ideally transformation of cells. The vectors permit the introduction of polynucleotides of the invention into host cells for the purpose of obtaining expression of the transcribable sequence in the host cell.

Vectors useful in performance of the invention may be viral or non-viral. Non-viral vectors include plasmids and episomal vectors, typically with an expression cassette for expressing a protein and human artificial chromosomes. Non-viral vectors useful for expression of transcribable sequences include pcDNA3.1/His, pEBVHis A, B & C and others available commercially.

Useful viral vectors include those based on retroviruses, adenoviruses, adenoassociated viruses, herpes virus, SV40, papillomavirus, HBP Epstein Barr virus, Vaccinia virus and Semliki Forest virus (SFV). Adenoviruses are preferred. These vectors are often made of two components, a modified viral genome and a coat encapsulating it. Viral vectors can also be naked in that they are lacking a coat.

Vectors may additionally comprise enhancer sequences as well as any other regulatory elements required for episomal maintenance, chromosomal integration or for high level transcription. Selectable markers and other identification sequences may also be useful components of a vector. Yet further sequences can be included to assist with conferring stability of the vector inside or outside the cell, targeting the vector for transfection of a given cell type or mediating cell entry. Particularly useful as part of viral vectors are ligands which bind to tumour cell surface specific markers.

Polynucleotides of the invention may be introduced into target cells by liposomes or immunoliposomes. The lipsome composition may selectively target the liposomes for fusion with certain cell types or tissues. The liposomes may contain binding proteins or ligands specific for cell surface markers specific for the target cell type thereby facilitating entry of the polynucleotides into the invention preferentially or exclusively into the desired tumour cells.

Other methods of delivery of polynucleotides of the invention into cells is by way of direct cell uptake or ballistics.

The invention also includes cells containing a polynucleotide, plasmid, vector, virus or preferably an adenovirus as described herein.

In another aspect the invention provides a method of killing a tumour cell comprising introducing a polynucleotide, plasmid, virus or adenovirus as described herein into the cell. The introduction may occur in vivo or in vitro. For example, where a vector is able to replicate and infect further cells, preferably cancer cells specifically, then cancer cells can be extracted from a patient, infected in vitro and then infected cells reintroduced back into the patient.

The invention therefore provides a method of treating or preventing cancer comprising administering an effective amount of a polynucleotide, plasmid, vector, virus or adenovirus as described herein to an individual.

In another aspect the invention provides a method of inhibiting expression of Skp 2 in a cell comprising introducing a polynucleotide, plasmid, vector, virus or adenovirus as described herein into the cell. The inhibition may be partial or total.

The invention also provides a pharmaceutical composition comprising a polynucleotide, plasmid, vector, virus or adenovirus as described herein.

Preferred compositions further comprise one or more pharmaceutically acceptable diluents, excipients or carriers, e.g. saline, buffered saline, water or dextrose. All forms of administration such as parenteral or oral are within the scope of the invention and the formulations may comprise other active agents, whether for simultaneous, separate or sequential administration, e.g. chemotherapeutic agents such as taxol for example.

Consequently, the invention includes the use of a polynucleotide, plasmid, vector, virus or adenovirus as described herein as a pharmaceutical.

The invention includes a polynucleotide, plasmid, vector, virus or adenovirus as described herein for use in the manufacture of a medicament for the treatment or prevention of cancer. The cancer preferably exhibits elevated skp2 promoter activity (i.e, SKP2 expression). The cancer may be one selected from cancers of the lung, stomach, breast, colon/rectum, uterine cervix, head or neck, for example.

In another aspect the invention provides a method for identifying a compound that modulates Skp 2 promoter activity comprising contacting a polynucleotide, plasmid, vector, virus or adenovirus as described herein with the compound and detecting a change in promoter activity. Promoter activity can be determined in an expression system, where the amount (or relative level compared to controls) of expression of a transcribable sequence is measured. Alternatively, the effect on transcription factor (e.g., E2F1) binding to the promoter or any other way of determining promoter activity can be employed. The compounds to be tested may be synthetic or naturally occurring.

An alternative method for identifying a compound that modulates Skp 2 promoter activity comprises contacting a transformed host cell which expresses a transcribable sequence under the control of an Skp 2 promoter with the compound and then measuring the amount of expression of the transcribable sequence.

In the aforementioned methods of identifying Skp 2 promoter modulatory compounds the expression system used to measure the amount of expression may be in vivo or in vitro, i.e. in the latter case the system may be a cell free system. The amount of expression may be determined by measuring the amount of the expressed product of the transcribable sequence, e.g. detection of a label integrated into the product or detection of a natural (or induced) property of the product, such as fluorescence, enzyme activity or ligand binding activity. A decrease in measured promoter activity indicates that the test compound is an inhibitor of SKP2 promoter activity.

The invention includes a method of inhibiting an Skp 2 promoter in a cell comprising introducing a polynucleotide, plasmid, vector, virus or adenovirus as described herein into the cell or administering a compound identified by the screening methods described herein.

As is apparent to one of ordinary skill in the art, the methods and materials described herein, although particularly useful in screening for anti-cancer agents and cancer therapeutics, are useful in any disease or disorder depending on inappropriate Skp2 promoter activity.

Preferred embodiments of the invention will now be described by way of specific examples and by reference to drawings in which:

FIGS. 1A, 1B and 1C show a 3720 base pair sequence of a linear DNA fragment comprising the Skp 2 promoter (SEQ ID NO:1).

FIG. 2 shows the DNA sequence of base pairs 2653 to 3024 of FIG. 1C and identifies particular regions of the Skp 2 promoter (SEQ ID NO:2).

FIG. 3 shows a map of the 5′ untranslated region (UTR) for murine Skp 2.

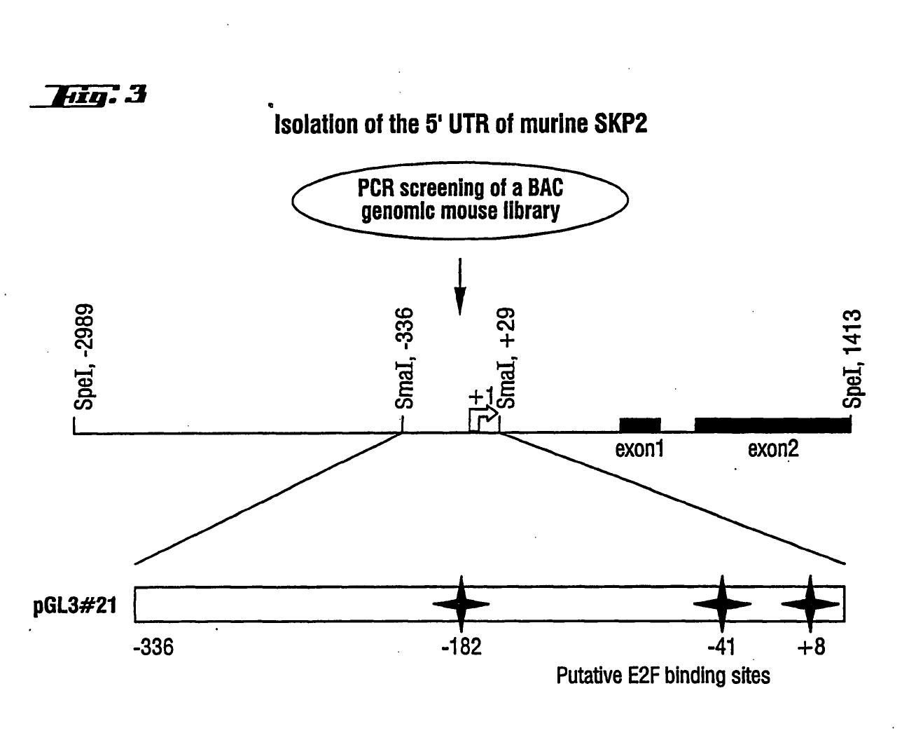

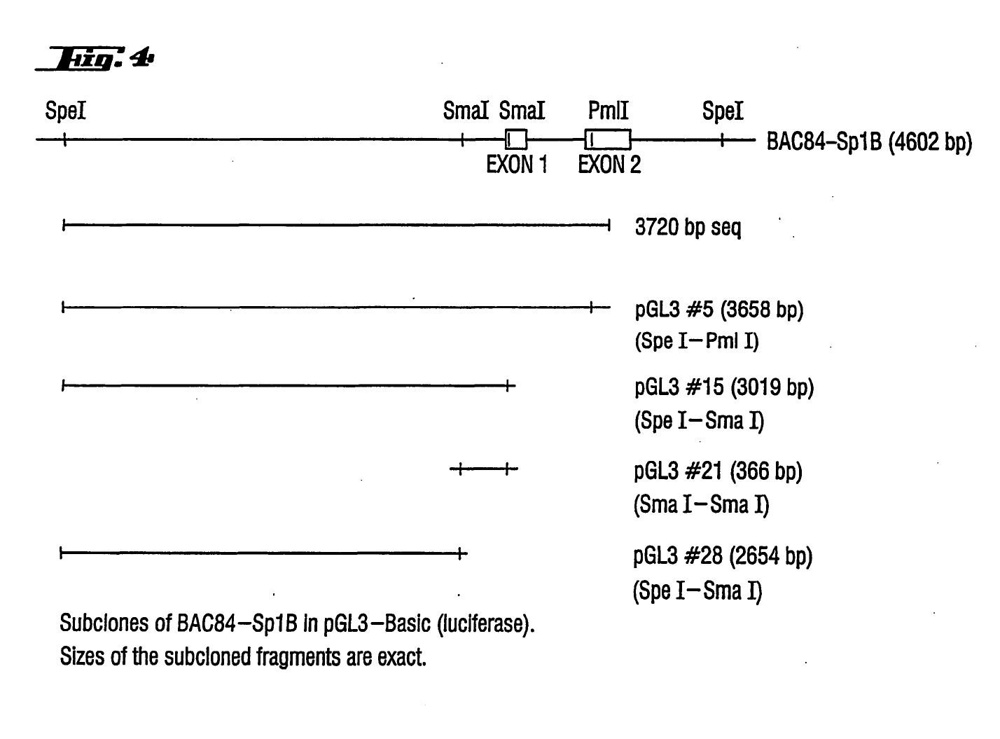

FIG. 4 shows maps of pGL3 plasmids comprising regions of the Skp 2 promoter.

The examples below are provided solely for illustrative purposes and are not to be found limiting to the appended claims.

### EXAMPLES

Cell culture. The cells used in the Examples below were: rat embryonic fibroblasts REF52, low passage number murine embryonic fibroblasts, HEK293 (ATCC CRL-1573), transformed human embryonic kidney cells, Cre8; ACHN renal adenocarcinoma (ATCC CRL 1611), human embryonic lung fibroblasts MRC5 (AG05965B), MRC5-SV40 (AG10076), IMR90 (IMR90), IMR90-SV40 (AG03204A) all originating from the NIA Aging Cell Culture Repository (Camden, N.J.), and human mammary epithelial cells HMEC originated from Clonetics BioWhittaker Europe. HMEC were cultured in Mammary Epithelial Cell Basal Medium complemented with supplements (bovine pituitary extract, human recombinant EGF, insulin, hydrocortisone and gentamycin) purchased from the supplier. Additional breast cells were SKBR-3 (malignant pleural effusion from breast adenocarcinoma, ATCC HTB 30), MDA-MB231 (pleural effusion from breast adenocarcinoma, ATCC HTB 26), BT474 (breast ductal carcinoma cells, ATCC HTB 20). RP-TEC were purchased from Clonetics BioWhittaker Europe and cultured in the medium recommended by the supplier. All cells except the HMEC and RPTEC were maintained in DMEM supplemented with 10% FCS (Gibco).

### Example 1

### Cloning of the mouse Skp 2 Promoter

The GI-H3 (5′-TAGGAAGCACCTTCAGGAGATTCC-3′; SEQ ID NO:3) and GI-H4 (5′-ACCTGGAAAGTTCTCCCGACTMG-3′; SEQ ID NO:4) oligonucleotide primers were used to screen a BAC (bacterial artificial chromosome) mouse genomic DNA library by PCR (FIG. 3). The genomic library is one made from embryonic stem cell DNA (Shizuya et al, (1992) PNAS 89: 8794-8797). Such DNA libraries are commercially available. The product of a PCR amplification of mouse genomic DNA using H3 and H4 primers is a 277 base pair fragment corresponding to exon 2 of the mouse Skp 2 gene.

Using a standard technique, DNA was prepared from the BAC clone found to comprise the 277 base pair fragment. The DNA of the BAC clone was digested with Spe I (FIG. 3) and subcloned into the pBluescript vector (Stratagene). The subclone containing H3 and H4 was identified and isolated and is termed BAC84-Sp1B. The clone was sequenced using external (T3 and T7, pBluescript vector specific) and internal (clone specific) oligonucleotides and standard techniques.

The internal oligonucleotides used for the sequencing of BAC84-Sp1B were:

| | |
|---|---|
| GI-H3: | |
| 5′-TAGGAAGCACCTTCAGGAGATTCC-3′ | (SEQ ID NO:3) |
| GI-H4: | |
| 5′-ACCTGGAAAGTTCTCCCGACTAAG-3′ | (SEQ ID NO:4) |
| GI-I1: | |
| 5′-TGCATGGTGTCCACTGATTCAGGA-3′ | (SEQ ID NO:5) |
| GI-I2: | |
| 5′-CATTCAAAACTCCTGAATCAGTGG-3′ | (SEQ ID NO:6) |
| GI-I5: | |
| 5′-GATTCCTGAAGACTTAATGACTGG-3′ | (SEQ ID NO:7) |
| GI-I6: | |
| 5′-CGCGTGAGGTGCGTCAGGGTCTGG-3′ | (SEQ ID NO:8) |
| GI-I7: | |
| 5′-CATTCTTTACTCTTCTTTCATGCC-3′ | (SEQ ID NO:9) |
| GI-I8: | |
| 5′-TATGTTAGATTGCATACACTTGTG-3′ | (SEQ ID NO:10) |
| GI-I9: | |
| 5′-TTTCCTAGAGAGAACCTTGTGCCC-3′ | (SEQ ID NO:11) |
| GI-J1: | |
| 5′-TGTGTGCCACCACTGCCCGTTACG-3′ | (SEQ ID NO:12) |
| GI-J2: | |
| 5′-AGTTGTGATGGACCCACATACAGC-3′ | (SEQ ID NO:13) |

BAC84-Sp1B has a total length of 4602 base pairs and contains exons 1 and 2 of the mouse Skp 2 gene, as well as about 3000 base pairs of upstream 5′ sequences (see FIG. 3). FIG. 2 (SEQ ID NO:2) shows the DNA sequence of a Sma I digest of the Spe I fragment shown in FIG. 3. The Sma I fragment is also shown in FIG. 3 and is termed pGL3#21. FIGS. 1A-1C show the DNA sequence of pBAC84G1 (which is a derivative of the Spe I fragment of FIG. 3, but with some of the coding region of exon 2 missing).

RACE (Rapid Amplification of cDNA Ends) analysis was used for the identification of the trancription start site. In brief, total RNA isolated from murine testis was reverse-transcribed with AMV-RT using a Skp2 specific primer hybridizing in exon 2 (5′CTCCCGACTMGCTTCGGCCGA3′; SEQ ID NO:14). Using the manufacturer's recommended procedure, the cDNA was purified (High Pure PCR purification columns, Roche) and tailed with an OligodT anchor (Roche). A first PCR round was performed using the OligodT anchor and a nested Skp2 specific primer (5′GAGCAGTCCATGTGGGATGT TCTCA3′; SEQ ID NO:15). A second PCR round was performed using the OligodT anchor and another nested Skp2 specific primer (5′CATCCCCACGT GAAGCTGGTGGT3′; SEQ ID NO:16). The PCR product were gel purified, digested with SalI, and subcloned into pBluescript (Stratagene). The RACE analysis yielded a dominant PCR fragment of about 250 base pairs, thereby identifying a major trancription start site.

Four different fragments of BAC84-Sp1B were subcloned into the pGL3-Basic vector (Promega) upstream of the luciferase gene in order to study the specific promoter activity of each of them. These clones are shown graphically in FIG. 4:
i) pGL3 #5—a SpeI-PmlI fragment extending from base 1 to base 3658 of BAC84-Sp1B.
ii) pGL3 #15—a Spe I-Sma I fragment extending from base 1 to base 3019 of BAC84-Sp1B.
iii) pGL3 #21—a 366 base pair Sma 1-Sma I fragment extending from base 2654 to base 3019 of BAC84-Sp1B.
iv) pGL3 #28—a Spe I-Sma I fragment extending from base 1 to base 2654 of BAC84-Sp1B.

pGL3#21 was constructed by digestion of BAC84-SP1B with Sma I and ligation of the fragment into pGL3-Basic vector (Promega). pGL3#21 was found to contain enough information to drive strong reporter gene expression and to be activated by E2F-1.

### Example 2

### Characterisation of pGL3#21 in MEFs. IMR90 and MRC5 Cells

Two different systems were used to identify the Skp 2 promoter. Primary mouse embryonic fibroblasts (MEFs) and their corresponding E1A/ras-transformed derivatives (MEF-E1A/ras) were transfected with Skp 2 promoter-luciferase reporter plasmids and promoter activity measured. The Skp 2 promoter displayed little reporter activity in MEFs but increased activity in the transformed derivatives.

Northern and Western blot analyses revealed that in the MEF-E1A/ras cells Skp 2 mRNA and protein levels were likewise enhanced suggesting that Skp 2 expression is affected by the E1A and ras oncoproteins. The former is well known to disrupt the pRB-pathway thereby liberating E2F.

A similar type of analysis was also performed in human cell lines. Specifically, Skp 2 promoter activity was determined, in parallel, in primary human lung fibroblasts IMR90 (or MRC5) and the corresponding SV40 large T antigen-transformed derivatives. Again Skp 2 promoter activity was low in the normal cells but very high in the transformed cell line, suggesting a potential linkage between Skp 2 promoter activity and transformed state.

In more detail, this activity of the promoter fragment subcloned as pGL3#2 was evaluated by transfecting cell lines such as MEF (murine embryonic fibroblasts), E1A+ras transformed MEF, human lung fibroblasts MRC5 (or IMR90) and SV40 transformed MRC5 (or IMR90) with 1 microgram pGL3#21. To optimise transfection efficiency of the non transformed cells, the commercial FUGENE reagent (Roche) was used according to the manufacturer's instructions. Typically, cells were grown in 24 well-dishes to 80% confluence. Tranfection complexes were formed using a FuGENET™6: DNA ratio of 3:1 (μl: μg). Efficiency of transfection between cell lines was normalized by co-transfecting a plasmid encoding β-galactosidase (0.1 microgram). Thus, 500 μg of total DNA comprising test plasmid (expressing luciferase) and normalizing plasmid (expressing lacz) in a ratio of 49:1 was added per well. Transfections were typically set in triplicates.

The transfected cell lines were then measured for luciferase activity 24 to 48 hrs following transfection. Briefly, cells were harvested 24 to 48 hours post-transfection and lysed in 0.1 M potassium phosphate pH 7.8, 0.1% Triton X-100, DTT 0.5 mM. Cleared supernatant was assayed for luciferase and lacz activities by chemiluminescence. Lacz activity was measured with a chemiluminescent reporter assay system (Galacto-LightTM, Tropix) using a 1,2-dioxetane substrate. Samples were measured in a luminometer (MicroLumat LB96P, EG&G Berthold) operated with the Winglow software. A representative increase of normalized Skp 2 promoter activity between transformed and non-transformed cells was typically at least 5-fold.

Endogenous levels of Skp 2 protein in the transformed cells was assayed using standard Western blotting protocols. A polyclonal antibody raised against full length Skp 2 (Lisztwan et al, (1998) EMBO J. 17: 368-383) was used. As secondary antibody, a commercially available (Amersham) horseradish peroxidase (HRP)-labelled anti-rabbit antibody was used. The enhanced chemoluminescence (ECL) detection kit (Amersham) was used.

Endogenous levels of Skp 2 mRNA were assayed using Northern blotting or by RT-PCR using following specific primers: 5′CCACCAGCTTCACGTGGGGA3′ (sense; SEQ ID NO:17) and 5′GCAGTCATTCTTTAGCATGA3′ (antisense; SEQ ID NO:18). The presence of Skp 2 mRNA gave a detectable band corresponding to a nucleic acid approximately 932 nucleotides in length. Increases in Skp 2 mRNA of between 3 to 8-fold can be seen between transformed and untransformed cells.

### Example 3

### E2F Binding Sites in the Skp 2 Promoter

Oligonucleotides corresponding to three of the four regions within 0.5 kb of the promoter sequence containing potential E2F sites bind readily to recombinant E2F in gel-shift assays in a specific manner. Gel shift assays with E2F can be carried out as described by Krek et al (1993) Science 262: 1557-1560. E2F-site mutant oligonucleotides failed to bind recombinant E2F in these DNA band shift assays. These results show that E2F has the capacity to directly activate the Skp 2 promoter. In more detail, four regions (FIG. 2) rich in potential E2F binding sites are to be found in the Skp 2 promoter Sma I fragment. These potential E2F binding sites were identified on the basis of their containing the core sequence SSCGC (where S equals C or G) of a ‘regular’ E2F binding site (TTTSSCGC), or being close to TTTSSCGC itself. The binding sites encompass four discrete regions; their numbering refers to the initial Spe I promoter fragment in which the Sma I subfragment spans nucleotides 2654 to 3019.

For ease of convenience only a single strand sequence is presented for each of the E2F binding sites below:

First Region Site:

2693GCTCCGGGAAAA2704 (SEQ ID NO:19): gel shift analysis showed no binding with baculovirus-expressed E2F-1/DP1.

Second Region Site:

2804MTCCCGCCCGGCGCCGCAGG2824 (SEQ ID NO:20) (underlined is a Sp1 binding site which may contribute to the basal activity of the promoter).

This mutated to: 2804MTCCCACCCGGCACCTCAGG2824 (SEQ ID NO:21): 1^{st} mutation, abolishes binding in gel shift analysis with baculovirus-expressed E2F-1/DP1. Promoter activity was reduced by at least 2-fold but not activation by E2F-1.

Similar results were achieved with the following other mutations:

| | |
|---|---|
| 2804AATCCCGCCCGGCGCCGCAGG2824 | (SEQ ID NO:22) |
| mutated to: | |
| 2804AATCCATCCCGGCACCTCAGG2824 | (SEQ ID NO:23) |
| 2804AATCCCGCCCGGCGCCGCAGG2824 | (SEQ ID NO:24) |
| mutated to: | |
| 2804AATCCCGCCCGGATCCGCAGG2824 | (SEQ ID NO:25) |
| 2804AATCCCGCCCGGCGCCGCAGG2824 | (SEQ ID NO:26) |
| mutated to: | |
| 2804AATCCCGCCCGGCGCATCAGG2824 | (SEQ ID NO:27) |

Third Region:

(SEQ ID NO:28)

2946AGAGCGGGATGCAGAACTCCGCG [symbol] GGTGG2978
mutated to:

(SEQ ID NO:29)

2946AGAGC-------ATGCAGAACTCCGCA [symbol] GGTGG2978

The underlined portion is like the E2F(a) binding site described in Hiyama et al. (1998) Oncogene 16: 1513-23. This mutation abolished binding of E2F-1. Promoter activity was reduced by about 2-fold and activation by E2F-1 suppressed.

The following oligonucleotides are used for finer mapping of the third region:

(SEQ ID NO:28)

2946AGAGCGGGATGCAGAACTCCGCGCGCGCGGTGG2978
mutated to:

(SEQ ID NO:30)

2946AGAGATGGATGCAGAACTCCGCGCGCGCGGTGG2978

(SEQ ID NO:28)

2946AGAGCGGGATGCAGAACTCCGCGCGCGCGGTGG2978
mutated to:

(SEQ ID NO:31)

2946AGAGCGGGATGCAGAACTCCGATCGCGCGGTGG2978:

(SEQ ID NO:28)

2946AGAGCGGGATGCAGAACTCCGCGCGCGCGGTGG2978
mutated to:

(SEQ ID NO:32)

2946AGAGCGGGATGCAGAACTCCGCGATCGCGGTGG2978:

(SEQ ID NO:28)

2946AGAGCGGGATGCAGAACTCCGCGCGCGCGGTGG2978
mutated to:

(SEQ ID NO:33)

2946AGAGCGGGATGCAGAACTCCGCGCGATCGGTGG2978

(SEQ ID NO:28)

2946AGAGCGGGATGCAGAACTCCGCGCGCGCGGTGG2978:
mutated to:

(SEQ ID NO:34)

2946AGAGCGGGATGCAGAACTCCGATCGCGCGGTGG2978

(SEQ ID NO:28)

2946AGAGCGGGATGCAGAACTCCGCGCGCGCGGTGG2978
mutated to:

(SEQ ID NO:35)

2946AGAGCGGGATGCAGAACTCCGCGCGATCGGTGG2978

Fourth Region:

| | |
|---|---|
| 2994AGTTCGCGGGTCC3006 | (SEQ ID NO:36) |
| mutated to: | |
| 2994AGTTAACGGGTCC3006: | (SEQ ID NO:37) |

No significant difference in signal was visualized indicating that promoter activity was not dramatically affected by this mutation to the fourth region. It did not abolish E2F-1 activation.

### Example 4

### Cotransfection of Cells with Skp Promoter-Luciferase Plasmid and Effector Plasmid Encoding E2F-1

293 cells, murine embryonic fibroblasts and human lung embryonic fibroblasts were cotransfected with the Skp 2 promoter—luciferase plasmid pGL3#21 (1 microgram) and the effector plasmid encoding E2F-1 or mutants thereof (0.2 microgram). (See Krek et al, (1995) Cell, 83: 1149-1158, and Krek et al, (1993) Science, 262: 1557-1560). A β-galactosidase expressing plasmid under the control of a CMV promoter was employed as a control for the cotransfection experiments. E2F-1 was seen to be a superior activator of the Skp 2 promoter. This activation was specific because E2F-1 mutants defective in either DNA binding or transcriptional activation failed to activate the Skp 2 promoter-luciferase reporter. The Skp 2 promoter represents a regulatory element that is overactive when the pRB-pathway is disrupted.

Several other promoters containing E2F binding sites have been characterized. In each case known to date (e.g. cyclin E, cyclin A or E2F-1 promoter), promoter activity is cell cycle regulated, being undetectable in quiescent cells but high as cells exit G1 and enter S phase (Weinberg R. A. (1996) Cell 85: 457-459.)

In FIGS. 1A-1C, the following transcription factor binding or interaction sites are identified: Yi, Ap1, Ap2, Sp1, NFkB, YY1 and Myb. Also, the Sma I restriction sites are shown. The first ATG codon is the putative start of the Skp 2 structural gene and thus the second ATG codon encodes an internal methionine. The sequence downstream of the first ATG codon is a partial coding sequence encompassing exon 1 and the start of exon 2 of the Skp 2 structural gene.

Also shown in FIG. 2 are four regions containing possible E2F binding sites. The first region does not physically interact with baculovirus-expressed E2F1 in gel shift assays. However, as shown above, the second, third and fourth are shown to bind E2F1 in gel shift assays indicating a possible role for these regions in promoter activity. In addition, mutation in the second and third regions decreased promoter activity, as well as suppressing E2F activation in the latter case, supporting an important role of the second and third regions in the level of promoter activity, and an important role of the third region in response to E2F. The Skp 2 promoter does not appear to have a TATA box.

### Example 5

### Activity of the Skp 2 Promoter does not Alter During the Cell Cycle

Two experiments were performed. In the first endogenous Skp 2 mRNA levels were measured throughout the cell cycle in IMR90 and MEF cells by RT-PCR, as described above. The cells were synchronised by serum starvation and then serum stimulated. No clear variation in Skp 2 mRNA levels was seen throughout the cell cycle in any of the cells studied.

In the second experiment, promoter activity from the cloned fragments pGL3#5, pGL3#1 5, pGL3#21 and pGL3#28 was evaluated by measuring the level of luciferase activity. Typically, groups of REF52 cells were transfected with 1 microgram of one of the Skp 2 constructs (pGL3#5, pGL3#15, pGL3#21 and pGL3#28), serum starved and then stimulated to leave quiescence by addition of serum. Cells were serum starved (growth arrested) by incubation for 72 hours in DMEM containing 0.1% FCS. Cells were serum stimulated by addition of FCS to a final concentration of 10% and harvested after 0 to 36 hours after stimulation.

Luciferase activity was evaluated at different time points (zero to 40 hours at 6-10 hour intervals) after serum stimulation. Again, no variation in luciferase activity was observed throughout the cell cycle. In contrast, in the same experiment, the cyclin A promoter displayed, as expected, cell cycle periodicity. Skp 2 promoter activity is, in stark contrast to all other known E2F-regulated promoters, not regulated during the cell cycle but rather Skp 2 promoter activity is dependent on the status of the cell, i.e. transformed versus untransformed. The Skp 2 promoter is therefore well suited for tumour cell-selective transgene expression.

### Example 6

### Construction of Skp 2-Based Adenoviral Vectors

The adenoviral vectors are derived from human adenovirus type 5 and are rendered replication deficient through deletion of the viral region E1 between nucleotides 554 and 3328 (numbering from the Ad5 sequence). Additionally, the E3 viral region is deleted between nucleotides 28592 and 30470.

The viral E1 region was replaced with a transgenic region carrying the following features:
(i) a murine Skp 2 promoter sequence, (i.e., the 366 bp Sma I fragment shown in FIG. 2 and FIG. 4) or, as control, the CMV promoter obtained from a commercial vector pRc/CMV (Invitrogen);
(ii) the cDNA of interest (for example, EGFP, from pEGFP-N1 vector, Clontech; luciferase, from pGL3-Basic vector, Promega; or human E2F-1, De Gregori et al., (1997) Proc. Natl. Acad. Sci. USA 94: 7245-7250); and
(iii) the polyadenylation signal from SV40 (nucleotides 2752 to 2534 in the simian virus 40 genome).

The transgenic region of the adenoviral vector is therefore flanked by adenoviral ITR sequences. It will be apparent to one of ordinary skill in the art that various other vectors comprising a Skp2 promoter can easily be designed.

Recombinant viruses were produced in CRE8 cells using the system described by Hardy et al. (1997) J. Virol 71: 1842-1849. A first step was the construction of so called “transfer vectors” for each adenovirus. Each transfer vector used was derived from the published parental vector pAdlox (Hardy et al, (1997) Supra).

Transfer vector for AdcMvEGFP:

An EGFP cDNA fragment flanked with HindIII and XbaI cloning sites was subcloned directionally into pAdlox. This plasmid is called pFMI4. (The 5′ ITR=Inverted Repeat Region is followed by a residual Lox P site (see Hardy et al Supra) upstream of the CMV promoter.

Transfer vector for Ad^{Skp2}EGFP:

pFMI4 was digested with Spe I and HindIII and religated, yielding pFMI6. The latter plasmid was digested with EcoRV and the Sma I Skp2 promoter fragment was inserted. This plasmid was called pFMI7. (The 5′ ITR is followed by a residual Lox P site (see Hardy et al Supra) upstream of the Skp2 promoter).

Transfer vector for Ad^{Skp 2}E2F-1:

pFMI7 was digested with NcoI and treated with Klenow to produce blunt ends. The E2F-1 cDNA was inserted as a blunt-ended BamHI-klenow treated insert, yielding pFMI8.

Transfer vector for Ad^{Skp2}luciferase:

pFMI7 was digested with NcoI and XbaI and ligated with luciferase cDNA fragment flanked with NcoI and XbaI sites.

Transfer vector for Ad^{Skp2}E2F-1/EGFP (Fusion Protein):

A fragment carrying the E2F-1 coding sequence minus the stop codon, and flanked by two NcoI sites is produced by PCR and subcloned into pFMI6 linearised with NcoI.

For production of the recombinant adenoviruses by homologous recombination in Cre8 cells, the transfer vectors were linearised with SfiI and cotransfected into Cre8 cells with purified viral DNA from a helper parental adenovirus called Ψ5 (Hardy et al, (1997) Supra).

Titers of recombinant adenoviruses were determined in terms of total particles or in terms of plaque forming units. In typical experiments, cell lines IMR90 and IMR90-SV40 or HMEC and breast cancer cell lines and cells (BT-474, MDA-MB231 and SK-BR3 cells) were infected with equal particle numbers (ranging from 2×10³ to 5×10⁴ particles/cell) of either Ad^{Skp2}EGFP or Ad^{CMV}EGFP. After 24-30 hrs infection, cells were either examined live using a fluorescence microscope, or used for FACS analysis of EGFP expression.

Fluorescence microscopy showed strong EGFP expression in transformed cells and breast cancer cells. The intensity of the signal was weaker in non-tranformed cells, and particularly faint in breast primary epithelial cells (HMEC).

FACS analysis showed that the intensity of EGFP expression was about 10-fold higher in transformed cells and breast cancer cells compared to primary or non-transformed counterparts.

Similar results can be found in other cell types including tumour cell lines derived from head and neck tumours when compared to their primary normal counterparts. Similar results are also to be found in nude mice experiments when biopsy samples are taken and injected into nude mice. So too when renal carcinoma cells (ACHN cells, CAKI-1 cells, 786-0 cells or A498 cells) and their primary counterparts (e.g., RP-TEC) are employed in such experiments.

### Example 7

### Administration of Adenoviral Vectors to Nude Mice Carrying Subcutaneous Tumours

Tumourigenic cells (e.g. renal carcinomal cell lines) are injected into the flank of nude mice. Tumours are permitted to grow in the mice to a size of about 10 mm. Ad^{Skp2}EGFP is injected into the mice. The expression of EGFP is monitored in sections derived from tumour tissue and surrounding normal tissues. Additionally Ad^{Skp2}EGFP or Ad^{CMV}EGFP are injected into the flank of a nude mouse or intravenously that does not contain any tumour to evaluate EGFP expression under these conditions. EGFP expression is readily obtained in tumours and this shows that the Skp 2 promoter can confer high level expression of the reporter gene EGFP in tumours grown in mice.

### Example 8

### Effect of E2F1 on Skp2 Promoter Activity in SBBR3 Cells

SKB3 cells were co-infected with Ad-^{Skp2}-EGFP and Ad-^{CMV}-E2F1 or Ad-^{CMV}-luciferase using 10⁴ particles/cell for each virus. Cells were harvested after 20 hours and EGFP expression was analyzed by FACS. Ectopic expression of E2F1 increases Skp2 promoter activity as demonstrated by increased EGFP expression. This effect is not observed when co-infection is performed with the control virus Ad-^{CMV}-luciferase.

Similar effects were seen with MEF cells. In brief, E2F-1 −/− MEFs at passage 1 were transfected in 24-well plates with 1.7 microgram of test Skp2 promoter plasmid expressing luciferase (pGL3#5 or pGL3#21), in the presence or absence of 340 ng of effector plasmid expressing E2F-1 (controls included co.transfection with PBSK). A lacZ expressing plasmid (100 ng) was included for normalization. Cells were harvested 24 hours post transfection and the relative values (luciferase/lacZ) determined. High relative expression was seen with both the Skp2 promoter constructs, pGL3#21 showing higher activity than pGL3#5. E2F-1 induction of promoter activity was seen at a low level for the E2F-1 promoter and at much higher levels for both pGL3#5 and pGL3#21 (greater than 5-fold 10-fold more, respectively, than the E2F-1 promoter).

### Example 9

### Construction of an Adenoviral Vector that Utilises the Skp 2 Promoter to Drive the Expression of an Apoptosis Inducing Gene

The special properties of the Skp 2 promoter-based viral vector make it ideal for the expression of an apoptosis gene, in particular E2F-1. An adenoviral vector is constructed that contains E2F-1 cDNA downstream of the Skp 2 promoter. The vector is referred to as Ad^{Skp 2}E2F-1. E2F-1 is used because deregulated E2F-1 expression is known to induce both p53-dependent and p53-independent apoptosis. In transient transfection experiments E2F-1 serves as a potent activator of the Skp 2 promoter (see Example 8). The effects of the recombinant adenoviral vector is assessed in tissue culture cells and then on tumours grown in mice.

The combination of Skp 2 promoter-driven E2F-1 expression and ionising radiation on cell proliferation and tumour growth in vivo is investigated. First, exponentially growing IMR90 and IMR90SV40Tag-transformed cells are infected with Ad^{Skp 2}E2F-1 or Ad^{Skp 2}EGFP and protein expression of E2F-1 and EGFP assessed by Western blotting. The proliferation rate and the apoptotic index of Ad^{Skp2}E2F-1-infected cells is determined and compared with Ad^{Skp2}EGFP-infected cells. Recombinant adenoviruses containing E2F-1 mutant derivatives, in particular DNA binding defective species of E2F-1 are included as controls in order to assess whether the effects seen are E2F-1-dependent.

Normal and transformed cells differ in their response to Ad^{Skp2}E2F-1. Ad^{Skp 2}E2F-1 alters the responsiveness of transformed cells to ionising radiation.

The minimal dose of ionising radiation required for cell death is determined. A panel of head and neck cancer cell lines are also tested. These cells can be characterized with respect to p53 status as well as their radiosensitivity. A significant proapoptotic effect of Ad^{Skp2}E2F-1 is found in transformed and tumour cells.

A similar proapoptotic effect of Ad^{Skp2}E2F-1 is found in subcutaneous tumours implanted into nude mice. Skp 2-promoter driven expression of E2F-1 is sufficient to inhibit, at least partially, the growth of tumours. The tumours reduce in size. Ad^{Skp2}E2F-1-injected tumours, in contrast to control injected tumours, regress in response to lower doses of ionising radiation. Lower doses of radiation than hitherto possible can therefore be used to achieve a therapeutic effect. Tumour shrinkage is found and this is due to enhanced apoptosis.

The various experiments described herein show the usefulness of Skp 2 promoter-based viral vectors as cancer therapies.

### Example 10

### Construction of an Adenoviral Vector that Utilises the Skp 2 Promoter to Drive Expression of Thymidine Kinase (TK)

An adenoviral vector of example 9 is constructed but instead of E2F-1, TK cDNA is employed. When administered in conjunction with intravenous injection of gancyclovir, the adenoviral vector Ad^{Skp 2}TK causes a reduction in tumour size compared to appropriate controls. In conjunction with irradiation treatment tumour regression is found to occur at lower doses of irradiation.

### Example 11

### Screening Assay that Utilises the Skp 2 Promoter

An adenoviral vector of example 6 (Ad^{Skp2}-EGFP) is used to infect SKBR breast cancer cells (10⁴ viral particles per cell) and the cells are treated with inhibitors of ErbB2, MAPK, P13K or cdk2. The various inhibitors can be tested in the micro to millimolar range, for example. After 12 hours, cells are harvested and EGFP expression is analysed by FACS. The cdk2 inhibitor (α-cdk2 at 10 mM) resulted in a strong decrease in EGFP expression (less than 5% of control levels).

The tumor specificity of this effect can be determined by infecting SKBR cells with a control vector, for example the adenoviral vector of example 6 comprising the CMV promoter (Ad^{CMV}-EGFP) (10⁴ viral particles per cell). The control cells are treated with the inhibitor as described above. The cdk2 inhibitor had no effect on EGFP expression from the CMV promoter construct indicating a differential response from the SKP2 and the CMV promoter.

This example illustrates a screening method using breast cancer cells and EGFP under the control of a SKP2 promoter, although it is apparent to one of ordinary skill in the art that other suitable cells or cell lines could be used, as well as other means for detecting SKP2 promoter activity (such as the use of various reporter genes or SKP2 itself). The test compounds can be selected to have certain properties or may be present in libraries of chemical, natural or other compounds.

All references referred to herein, as well as priority application GB 001571.8 filed Jun. 23, 2000, are hereby incorporated by reference as if each were referred to individually.

1. A polynucleotide comprising an Skp 2 promoter sequence.

2. A polynucleotide as claimed in claim 1, wherein the promoter sequence comprises at least 15, optionally at least 25, optionally at least 50, optionally at least 100, or optionally at least 500 or a sequence hybridisable thereto under stringent conditions.

3. A polynucleotide as claimed in claim 1 or claim 2, wherein the sequence comprises no more than 10 kb, optionally no more than 5 kb, optionally no more than 1 kb.

4. A polynucleotide as claimed in any of claims 1 to 3 comprising a sequence as set forth in SEQ ID NO:1 or SEQ ID NO:2, or a fragment thereof, or a sequence hybridisable thereto under stringent conditions.

5. A polynucleotide or fragment as claimed in claim 4 having at least 60%, preferably at least 75%, more preferably at least 90%, even more preferably at least 95%, most preferably at least 99% homology with all or parts of SEQ ID NO:1 or SEQ ID NO:2.

6. A polynucleotide as claimed in any of claims 1 to 5, further comprising a transcribable sequence operably linked to the promoter sequence.

7. A polynucleotide as claimed in claim 6, wherein the expressed product of the transcribable sequence causes cell death or inhibits the division or viability of cells preferably the transcribable sequence is a pro-apoptotic gene.

8. A polynucleotide as claimed in claim 6 or claim 7, wherein the expressed product of the transcribable sequence is a toxin.

9. A polynucleotide as claimed in any of claims 4 to 6, wherein the expressed product of the transcribable sequence increases the sensitivity of tumour cells to antitumour agents or treatments, e.g. radiotherapy or chemotherapy.

10. A polynucleotide as claimed in claim 9, wherein there is a synergistic effect between the expressed product and the antitumour agent or treatment in terms of killing cells or inhibiting their division or viability.

11. A polynucleotide as claimed in any of claims 6 to 10 comprising a transcribable sequence which encodes a reporter protein or peptide.

12. A polynucleotide as claimed in claim 11, wherein the reporter is selected from one or more of an enzyme, a ligand binding protein, a fluorescent protein or a phosphorescent protein.

13. A polynucleotide as claimed in claim 12, wherein the reporter protein is selected from firefly luciferase, beta-glucuronidase, chloramphenicol acetyl transferase, green fluorescent protein, enhanced green fluorescent protein or human secreted alkaline phosphatase.

14. A polynucleotide as claimed in claim 6 or claim 7, wherein the transcribable sequence is an antisense sequence.

15. A polynucleotide antisense to a polynucleotide as claimed in any preceding claim.

16. A genetic construct comprising a polynucleotide as claimed in any of claims 1 to 15.

17. A plasmid comprising a polynucleotide as claimed in any of claims 1 to 15.

18. A vector comprising a polynucleotide as claimed in any of claims 1 to 15.

19. A vector as claimed in claim 18 being a virus, preferably an adenovirus.

20. A cell containing a polynucleotide of any of claims 1 to 14, a plasmid of claim 17, or a vector of claim 18 or claim 19.

21. A method of killing a tumour cell comprising introducing a polynucleotide comprising an Skp 2 promoter sequence, or a plasmid comprising a polynucleotide comprising an Skp 2 promoter sequence, or a vector comprising a Polynucleotide comprising an Skp 2 promoter sequence into the cell.

22. A method as claimed in claim 21, wherein the polynucleotide or vector is introduced into the cell in vitro.

23. A method of treating cancer comprising administering an effective amount of a polynucleotide comprising an Skp 2 promoter sequence, or a plasmid comprising a polynucleotide comprising an Skp 2 promoter sequence, or a vector comprising a polynucleotide comprising an Skp 2 promoter sequence to an individual.

24. A method of inhibiting expression of Skp 2 in a cell comprising introducing a polynucleotide comprising an Skp 2 promoter sequence of any of claims 1 to 15, or a plasmid comprising a polynucleotide comprising an Skp 2 promoter sequence of claim 17, or a vector comprising a Polynucleotide comprising an Skp 2 promoter sequence into the cell.

25. A pharmaceutical composition comprising a polynucleotide of any of claims 1 to 15, or a plasmid of claim 17, or a vector of claim 18 or claim 19.

26. A composition as claimed in claim 25, further comprising one or more pharmaceutically acceptable diluents, excipients or carriers.

27. The use of a polynucleotide of any of claims 1 to 15, or a plasmid of claim 17, or a vector of claim 18 or claim 19 as a pharmaceutical.

28. A polynucleotide as claimed in any of claims 1 to 15, or a plasmid of claim 17, or a vector as claimed in claim 18 or claim 19 for use in the manufacture of a medicament for the treatment of cancer.

29. A use as claimed in claim 28, wherein the cancer is one selected from cancers of the breast, head or neck.

30. A method for identifying a compound that modulates Skp 2 promoter activity comprising contacting a polynucleotide of any of claims 1 to 15 with the compound, subjecting the polynucleotide to an expression system and measuring the amount of expression of the transcribable sequence.

31. A method as claimed in claim 30, wherein the expression system is a cell free system.

32. A method for identifying a compound that modulates Skp 2 promoter activity comprising contacting a cell of claim 20 which expresses a transcribable sequence with a compound and measuring the amount of expression of the transcribable sequence.

33. A method as claimed in any of claims 30 to 32, wherein the amount of expression is determined by measuring the amount of activity of expressed product of the transcribable sequence.

34. A method of inhibiting an Skp 2 promoter in a cell comprising introducing a polynucleotide comprising an Skp 2 promoter sequence.
